(19) United States
(12) Patent Application Publication
(10) Pub. No.: US 2002/0068280 A1
(43) Pub. Date: Jun. 6, 2002

(54) Compositions and methods for DNA purification from whole blood

(76) Inventor: Jeff Fairman, Mountain View, CA (US)

Correspondence Address: RUTAN & TUCKER, LLP, P.O. BOX 1950, COSTA MESA, CA, 92628-1950, US

(21) Appl. No.: 09/732,105

(22) Filed: Dec. 6, 2000

(51) Int. Cl. C12Q 1/68; C07H 21/04

(52) U.S. Cl. 435/6; 435/270; 536/25.41

(57) ABSTRACT

Methods and compositions for isolation of genomic deoxyribonucleic acid from whole blood employ three distinct aqueous solutions, all of which are substantially free from chaotropic salts and organic solvents. Genomic DNA isolated according to the inventive subject matter is substantially free of polymerase inhibitors and of sufficient quality for quantitative PCR.

### FIELD OF THE INVENTION

The field of the invention is purification of nucleic acids.

### BACKGROUND OF THE INVENTION

Most nucleic acids, and particularly genomic DNA (deoxyribonucleic acid) are valuable starting materials for numerous cloning techniques, and various approaches are known in the art to obtain isolated and purified genomic DNA. Typically, a biological material is disintegrated to form a crude extract from which the genomic DNA is subsequently adsorbed onto a solid phase (e.g., anion exchange resin or silica). However, all or almost all of the known solid-phase based procedures include an organic solvent for elution and/or washing of the adsorbed DNA. Such solvents are often flammable, expensive, and difficult/expensive to dispose of when properly discarded. Moreover, safety and environmental problems are compounded in some techniques by addition of chaotropic salts to the sample to disintegrate cells and/or inactivate nucleases.

Although known DNA isolation procedures yield relatively clean DNA preparations as judged from their OD_{260/280} ratio, most downstream applications with relatively high sensitivity to impurities, particularly quantitative PCR (qPCR), require additional clean-up to remove polymerase inhibitors inherently present in most DNA samples isolated by such procedures. For example, Bourke et al. report [Bourke, M. T., Scherczinger, C. A., Ladd, C., and Lee, H. C. NaOH treatment to neutralize inhibitors of Taq polymerase; J Forensic Sci. 1999; 44(5):1046-50] that polymerase inhibitors may be removed by alkaline washing of the isolated DNA on a microfilter. Although alkaline washing tends to effectively remove impurities in the DNA sample, the NaOH protocol is not advised by Burke where the quantity of DNA is limited, since Burke's alkaline treatment results in a significant loss of DNA.

In another example published by Al-Soud et al. [Al-Soud, W. A., Jonsson, L. J., Radstrom, P. Identification and characterization of immunoglobulin G in blood as a major inhibitor of diagnostic PCR; J Clin Microbiol 2000 Jan;38(1):345-50], the authors identify antibodies and antibody fragments as potential polymerase inhibitors, which is especially problematic when the sample for a qPCR is blood or derived from blood. Consequently, at least one of the sample and the blood or blood derivative needs to be pretreated to remove the immunoglobulin fraction (e.g., protease digest, protein A, or protein G chromatography), thereby adding purification steps that prolong the isolation procedure, and potentially adding substantial cost.

In a further example, Giambernardi et al report [Giambernardi, T. A., Rodeck, U., Klebe, R. J.; Bovine serum albumin reverses inhibition of RT-PCR by melanin; Biotechniques 1998; 25(4):564-6] that the RT-PCR inhibitor melanin can be efficiently removed by simple addition of bovine serum albumin (BSA). Although addition of BSA is a relatively simple step to remove an impurity, Giambernardi's protocols tends to be limited to removing specific impurities (i.e., melanin) from specific samples (i.e., benign and malign melanocytes).

Worse yet, all or almost all additional manipulation steps to clean up previously isolated genomic DNA tend to shear the genomic DNA, which is often undesirable in many subsequent applications. To circumvent at least some of the problems with additional clean-up steps, genomic DNA can be prepared from clarified crude extracts using CsCl density gradient centrifugation. CsCl procedure typically yields highly pure and high molecular weight genomic DNA, however, has several significant disadvantages. For example, CsCl density gradient centrifugation requires ultracentrifugation, which translates into significant operating cost (e.g., limited sample capacity, high cost of equipment, etc.). Moreover, automation of the isolation of genomic DNA is typically difficult to implement.

In yet another alternative method, pressure desorption from a solid phase is employed to isolate highly purified DNA as described in U.S. Pat. No. 6,111,079 to Lugharn Jr., et al. (Aug. 29, 2000). Lugharn's procedure can be automated in a relatively simple manner, and may advantageously be performed with a minimal number of aqueous buffers. However, Lugharn's procedure requires specialized equipment to operate at pressures of 100,000 psi, and even higher, to desorb the nucleic acid from the solid phase, thereby significantly increasing operating and maintenance costs.

Although various method of isolating genomic DNA from biological material are known in the art, all or almost all of them suffer from one or more disadvantage. Therefore, there is still a need to provide methods and compositions for improved isolation of genomic DNA from biological material.

### SUMMARY OF THE INVENTION

The present invention is directed to methods and compositions for isolation of genomic DNA from biological fluids, particularly blood, in a simple procedure employing aqueous solvents without use of organic solvents and chaotropic salts. The isolated genomic DNA is substantially free of polymerase inhibitors.

In one aspect of the inventive subject matter, the biological fluid comprises whole blood having at least one red blood cell, and one white blood cell including DNA and protein. A first aqueous solution lyses the red blood cell, a second aqueous solution is used to wash the white blood cell, a third aqueous solution lyses the white blood cell, and a fourth aqueous solution precipitates the protein.

In another aspect of the inventive subject matter, the first and second aqueous solutions are identical and comprise at least two different inorganic salts, preferably ammonium chloride and sodium bicarbonate, the third aqueous solution comprises a detergent, preferably sodium lauryl sulfate, and the fourth aqueous solution comprises an organic salt, preferably ammonium acetate.

In a further aspect of the inventive subject matter, a kit for purifying genomic DNA from whole blood comprises the first, third and fourth aqueous solution, and optionally further comprises isopropanol for precipitation of the genomic DNA.

Various objects, features, aspects and advantages of the present invention will become more apparent from the following detailed description of preferred embodiments of the invention, along with the accompanying drawing.

### BRIEF DESCRIPTION OF THE DRAWING

FIG. 1 is a flow chart of an exemplary method of isolating genomic DNA from a biological fluid according to the inventive subject matter.

FIG. 2 is a perspective schematic view of a kit for purifying genomic DNA from a biological fluid according to the inventive subject matter.

### Detailed Description

As used herein, the term “genomic DNA” refers to double stranded deoxyribonucleic acid that constitutes the genome of an organism, and that is passed along in equal proportions to the daughter cells as a result of a cell division of a parental cell. The term “genome” as used herein means the total set of genes carried by an individual or cell, which define the individual or cell as belonging to a particular genus and species. For example, DNA in a chromosome is regarded genomic DNA under the scope of this definition, because a chromosome is part of the genome of an organism, and is passed along in equal proportions to F1 cells as a result of a cell division of a P1 cell. In contrast, a recombinant plasmid, phagemid, or artificial chromosome is not considered genomic DNA under the scope of this definition, because the recombinant plasmid, phagemid, or artificial chromosome is not part of the genome of an organism, and is typically not passed along in equal proportions to F1 cells as a result of a cell division of a P1 cell.

As also used herein, the term “red blood cell” refers to a fully or partially differentiated erythrocyte, which may or may not have a micronucleus. The term “white blood cell” as used herein refers to all blood cells other than red blood cells, and particularly includes macrophages, B- and T-lymphocytes, monocytes, neutrophiles, eosinophiles, and basophiles.

As further used herein, the term “protein” refers to any polypeptide of natural or recombinant origin that has been synthesized in a red or white blood cell. Contemplated polypeptides include secreted, soluble, and transmembranous proteins, which may have posttranslational modifications such as glycosylation, acylation, methylation, phosphorylation, sulfation, prenylation, etc.

As still further used herein, the term “lysing” a cell refers to any form of permanent physical and/or chemical disintegration of the structure of the cell. For example, cracking a cell by osmotic pressure, or dissolving the fluid mosaic structure of a cell membrane by detergents is considered lysing a cell under the scope of this definition. In contrast, electroporation is not consistent with the term lysing, because electroporation typically establishes structural disintegration only for short periods.

As also used herein, the term “chaotropic salt” refers to an organic or inorganic salt that disrupts the configuration of the hydration spheres surrounding a molecule (typically a protein), thereby disrupting salt bridge formation and dielectric interactions. Particularly contemplated chaotropic salts include potassium iodide, sodium iodide, potassium thiocyanate, urea, and guanididium hydrochloride.

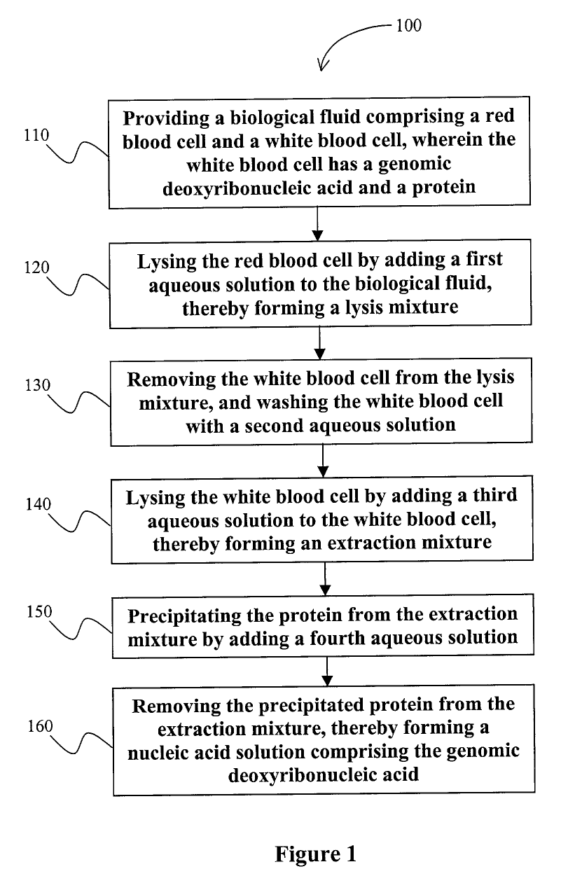

The inventors surprisingly found that genomic DNA substantially free of polymerase inhibitors can be isolated from a biological fluid following a simple protocol employing only aqueous solutions. An exemplary protocol is depicted in FIG. 1, in which the isolation method 100 has a first step 110, in which a biological fluid comprising a red blood cell and a white blood cell is provided, and wherein the white blood cell has a genomic deoxyribonucleic acid and a protein. In step 120, the red blood cell is lysed by adding a first aqueous solution to the biological fluid, thereby forming a lysis mixture, and in another step 130, the white blood cell is removed from the lysis mixture and is washed with a second aqueous solution. In step 140, the white blood cell is lysed by adding a third aqueous solution to the white blood cell, thereby forming an extraction mixture, and in a subsequent step 150, the protein is precipitated from the extraction mixture by adding a fourth aqueous solution. In a further step 160, the precipitated protein is removed from the extraction mixture, thereby forming a nucleic acid solution comprising the genomic deoxyribonucleic acid.

In a particularly preferred aspect of the inventive subject matter, 5 ml of fresh drawn whole blood are dispensed into a 15 ml centrifuge tube. 8 ml of the first aqueous solution (8.28 g ammonium chloride, 0.84 g sodium bicarbonate, and 500 μl of 0.2 M ethylenediaminetetraacetic acid (EDTA) per liter deionized autoclaved water) are added to the blood to lyse the red blood cells, and the lysis mixture is repeatedly pipetted up and down or vortexed to ensure adequate mixing. After incubation at room temperature for 15 minutes, the lysis mixture is centrifuged at 2500×g for 2 minutes. The supernatant is discarded without disturbing the pellet, and 5 ml of a second aqueous solution (identical to the first aqueous solution) are added to the pellet and pipetted up and down 3 times (or vortexed) to ensure that the pellet is resuspended. The resuspended pellet is then incubated at room temperature for 15 minutes, and again centrifuged at 2500×g for 2 minutes. The supernatant is discarded without disturbing the pellet, and 2 ml of the third aqueous solution (1 g of SDS per liter deionized autoclaved water) are added to the pellet to form an extraction mixture. The extraction mixture is pipetted up and down 10 times (or vigorously vortexed) to ensure that the pellet is dissolved and the cells are ruptured. 2 ml of the fourth aqueous solution (385.4 g ammonium acetate per liter deionized and autoclaved water) are added to the extraction mixture and the suspension is vortexed for 30 seconds to ensure adequate mixing. After mixing, the extraction mixture is centrifuged at 3000×g for 15 minutes, and the supernatant (i.e. the nucleic acid solution) is transferred to a fresh tube.

With respect to the biological fluid, it should be appreciated that numerous biological fluids other than fresh drawn whole blood are also contemplated, and alternative fluids include fluids comprising cells from an in vivo or an in vitro origin. For example, where a blood sample is not fresh drawn, stored, frozen, cooled or otherwise preserved blood samples are contemplated. In another example, whole blood from which at least one component has been extracted for commercial, therapeutical or analytical purposes (e.g., fibirin or platetlets) is contemplated an appropriate biological fluid. Alternatively, suitable biological fluids may also include additional reagents, including coagulation inhibitors, buffers, chelators, organic and inorganic acids and/or bases, antibodies, etc.

In further alternative aspects, appropriate biological fluids also include various non-blood fluids, (e.g., a bone marrow suspension), and in a further example, it is contemplated that suitable fluids may also include cell suspensions of wild-type and recombinant cells from an suspension and/or adhesion culture. While it is generally contemplated that the biological fluid is derived from a mammal, preferably a human, alternative sources for the biological fluid include vertebrates and invertebrates.

It should further be appreciated that the genomic DNA in the white blood cell is not restricted to a particular configuration, and all physiological configurations, including heterochromatin, euchromatin, and condensed chromosomes are contemplated. Likewise the size of the genomic DNA may vary substantially, however it is preferred that the size is at least 25 kBp, preferably more than 100 kBp, more preferably more than 1 MBp, and most preferably more than 100 MBp.

In yet other alternative aspects of the inventive subject matter, the composition of the first aqueous solution need not be restricted to about 150 mM NH₄Cl, about 100 mM NaHCO₃, and about 0.1 mM EDTA, and various alternative compositions are also contemplated. Suitable compositions include one or more inorganic and/or organic salts, which may or may not have additional chelating agents. For example, where only one salt is preferred, NH₄CO₃ may be employed at a concentration of about 200-300 mM. Alternatively, where an organic salt is preferred, ammonium acetate may be advantageously utilized which may be buffered to the appropriate pH. Similarly, the molarities of the components need not be limited to the indicated molarities, so long as the first aqueous solution lyses the red blood cells with at least 10-fold, preferably at least 100-fold, and more preferably at least 1000-fold greater selectivity than the white blood cells. It should furthermore be appreciated that the chelating agent may vary considerably, and suitable chelators include bidentate, tridentate, and tetradentate chelators. However, especially preferred chelators are EDTA, EGTA, and desferoxamine. Likewise, suitable concentrations of contemplated chelators may very substantially and particularly appropriate concentrations are in the range of about 10 μM to about 10 mM.

While it is generally contemplated that the red blood cells lyse upon mixing with the first aqueous solution by virtue of the composition of the first aqueous solution, it should also be appreciated that additional chemical and/or physical steps may be performed to assist in lysis of the red blood cells. For example, contemplated additional steps include physical, chemical, mechanical manipulations, and all reasonable combinations thereof, and particularly contemplated additional steps are incubation at decreased (i.e., below room temperature) temperature, incubation at increased (i.e., above room temperature) temperature, sonication, saponification, osmotic shock, etc.

Thus, it is contemplated that the lysis mixture may require additional incubation, and especially contemplated incubation times include periods of between about several seconds and 30 minutes. However, where appropriate, incubation of the lysis mixture may be entirely omitted. Alternatively incubation of the lysis mixture may also extended beyond 30 minutes where time is not of the essence, or substantially complete lysis of the red blood cells (i.e., more than 95%) is achieved with an incubation longer than 30 minutes. Incubation may further include additional reagents, including enzymes, detergents, antibodies, etc.

With respect to the step of removing the white blood cells, it is preferred that the lysis mixture is centrifuged at about 2500×g for approximately 2 minutes. However, it should be appreciated that centrifugation may also be performed at different centrifugal forces. For example, where the white blood cells are relatively fragile, centrifugal forces of less than 2500×g are especially contemplated, including 2500×g to 500×g, and less. On the other hand, where it is desirable that the time of the centrifugation is reduced, centrifugal forces of more than 2500×g are contemplated, including 2500×g to 4000×g, and more. Consequently, it should be recognized that the time of centrifugation may vary substantially, and suitable times will typically depend on the particular composition of the sample (e.g., viscosity), the applied centrifugal force, and the desired degree of separation. It should further be recognized that the step of removing the white blood cells from the lysis mixture generally refers to separating the white blood cells from the lysis mixture. Consequently, the white blood cells may be removed from the lysis mixture, or the lysis mixture can be removed from the white blood cells. In still further alternative aspects of the inventive subject matter, separation methods other than centrifugation are also contemplated, and particularly preferred alternative methods include filtration, separation with magnetic beads capturing the white blood cells, and antibody-mediated coagulation/sedimentation of the white blood cells.

While it is generally preferred that the cells are washed only once with the second aqueous solution (which is the same as the first aqueous solution), multiple washes, including washes with aqueous solutions other than the second aqueous solution are also contemplated. The term “washing” cells as used herein means that cells are suspended in a washing fluid by repeatedly pipetting, vortexing, or otherwise mixing to form a cell suspension, and that the cells are then removed (typically by centrifugation) from the washing fluid. For example, where remaining traces of hemoglobin are still present in the suspension of washed cells, additional washes (i.e., a third wash, a fourth wash, or even a fifth wash, etc.) may be performed to remove the contaminant. Still further, the second aqueous solution need not be limited to be the same as the first aqueous solution, and various alternative aqueous solution may be employed for washing the cells, so long as the second aqueous solution does not lyse the white blood cells to a significant degree (no more than 25% of all white blood cells, preferably no more than 10% of all white blood cells, more preferably no more than 5% of all white blood cells, and most preferably no more than 2% of all white blood cells). Contemplated alternative second aqueous solutions include buffered and unbuffered aqueous solutions, and particularly contemplated alternative wash solutions have osmotic pressure, ionic strength and pH similar to the first aqueous solution.

In yet another alternative aspect of the inventive subject matter, the white blood cells are lysed with a solution other than the third aqueous solution, and particularly contemplated alternative third aqueous solutions comprise an ionic detergent, a non-ionic detergent, a zwitterionic detergent, or any reasonable combination thereof. For example, where ionic detergents are particularly undesirable, non-ionic detergents such as Triton X-100 (NP40), octylglycoside, or Tween 20 may be utilized. On the other hand, where low cost is especially relevant, ionic detergents such as deoxycholic acid, alkylsulfonic or phosphonic acids are particularly contemplated. Alternatively, zwitterionic detergents like CHAPS and N-Dodecyl-N,N-dimethyl-3-ammonio-1-propane sulfonate may be useful where protein aggregation is to be avoided.

While it is generally contemplated that the white blood cells lyse upon mixing with the third aqueous solution by virtue of the composition of the third aqueous solution, it should also be appreciated that additional chemical and/or physical steps may be performed to assist in lysis of the white blood cells. For example, contemplated additional steps include physical, chemical, mechanical manipulations, and all reasonable combinations thereof, and particularly contemplated additional steps are incubation at decreased (i.e., below room temperature) temperature, incubation at increased (i.e., above room temperature) temperature, sonication, saponification, osmotic shock, etc.

Thus, it is contemplated that the extraction mixture may require additional incubation, and especially contemplated incubation times include periods of between about several seconds and 30 minutes. However, where appropriate, incubation of the extraction mixture may be entirely omitted. Alternatively incubation of the extraction mixture may also extended beyond 30 minutes where time is not of the essence, or substantially complete lysis of the white blood cells (i.e., more than 95%) is achieved with an incubation longer than 30 minutes. Incubation may further include additional reagents, including enzymes, detergents, antibodies, etc.

While it is generally preferred that the proteins present in the extraction mixture are directly precipitated from the extraction mixture, it should also be appreciated that additional intermediate steps may be included to clarify the extraction mixture from cellular debris. Particularly contemplated suitable intermediate steps include centrifugation, and filtration.

Regardless of the presence of an additional intermediate step, it is contemplated that addition of the fourth aqueous solution will result in substantially complete precipitation of the proteins present in the extraction mixture. Thus, it is contemplated that the chemical composition of the fourth aqueous solution need not be restricted to an approximately 5M aqueous solution of ammonium acetate, and alternative solutions include organic salts, inorganic salts, and all reasonable combinations thereof, so long as alternative fourth aqueous solutions will substantially completely precipitate the protein present in the extraction mixture. For example, especially contemplated suitable fourth aqueous solutions comprise ammonium sulfate, sodium chloride, and trichloroacetic acid individually, or in combination with ammonium acetate.

It should further be appreciated that the precipitation of the protein from the extraction mixture will typically not require an incubation step to allow the precipitation run to completion. However, it is contemplated that (where appropriate) additional incubation may also be included into the protocol. With respect to the incubation conditions for precipitation of the protein, the same considerations as for previously described incubations (supra) apply.

With respect to the step of removing the precipitate from the extraction mixture, it is generally preferred that the precipitate is removed by centrifugation for about 15 minutes at approximately 3000×g. It should be recognized, however, that the centrifugation conditions may vary considerably, and that a particular time and centrifugal force applied to the extraction mixture will typically depend on the degree of precipitation and coagulation, and on the amount of precipitated protein. Thus, alternative centrifugation times may be between about 15-5 minutes, and less, but also between 15 and 30 minutes, and more. Similarly, the centrifugal force may be less than 3000×g (e.g., between about 3000×g and about 1500×g and less), but also more than 3000×g (e.g., between about 3000×g and about 5000×g, and more). It is further contemplated that the extraction mixture may be cooled during the centrifugation where appropriate. It should further be recognized that the step of removing the precipitated protein from the extraction mixture generally refers to separating the precipitated protein from the extraction mixture. Consequently, the precipitated protein may be removed from the extraction mixture, or the extraction mixture may be removed from the precipitated protein. Alternatively, the precipitate may be removed from the extraction mixture by methods other than centrifugation, and particularly preferred methods include filtration.

It should generally be appreciated that the particular volumes of the biological fluid, first, second, third, and fourth aqueous solutions need not be limited to the volumes as indicated in the preferred aspect of the inventive subject matter, but may vary considerably so long as genomic DNA that is substantially free from polymerase inhibitors can be prepared from the biological fluid. It should generally be appreciated that where smaller volumes of the aqueous solutions are desirable, the concentration of the ingredients for the aqueous solutions may be increased. Likewise, higher volumes may be employed where the solutions have a lower concentration of their respective ingredients.

Alternatively, where only minor amounts of genomic DNA are required (or where only limited amounts of biological fluid is available), the volume of the biological fluid may be less than 5 ml (e.g., approximately 5 ml to 0.5 ml, and less). On the other hand, where more genomic DNA is required for subsequent steps, volumes of more than 5 ml, including 5 ml-50 ml, and more, are contemplated. Consequently, the volume of the first aqueous solution may be more or less than 8 ml, and will typically depend on the volume of the biological fluid. Likewise, the volume of the aqueous solution for the wash step may vary substantially, and may decrease as the number of wash steps increases. In further alternative aspects, the volume of the third aqueous solution may vary, so long as the third aqueous solution lyses at least a portion of the white blood cells. Similarly, the volume of the fourth aqueous solution need not be restricted to 2 ml, and appropriate volumes will generally be in the range of 0.2 ml and less to 20 ml, and more.

With respect to the isolated genomic DNA it should be appreciated that the size and integrity will typically depend on the particular protocol, however, it is preferred that the size of the isolated genomic DNA is at least 20 KBp, preferably greater than 50 KBp, more preferably greater than 75 KBp, and most preferably greater than 100 KBp. It is still further contemplated that the isolated genomic DNA is substantially free from a polymerase inhibitor. The term “genomic DNA substantially free from a polymerase inhibitor” as used herein means that when the genomic DNA is employed as a template in a qPCR reaction, amplification of a target sequence defined by a primer pair with opposite polarity (i.e., 3′—OH of both primers facing the stretch of DNA to be amplified) reproducibly produces the target sequence product in a logarithmic manner over at least 3 orders of magnitude independent of the particular sequence of the target sequence.

In an especially contemplated aspect of the inventive subject matter, the isolated genomic DNA is precipitated from the nucleic acid solution by adding to 4 ml of isopropanol to the tube and inverting about 20 times. After about 5 minutes at room temperature for 5 minutes, the precipitate is concentrated by centrifugation at 3000×g for 15 minutes, the supernatant removed, and the pellet is air-dried for about 10 minutes. Alternatively, the DNA obtained from the nucleic acid solution may be concentrated or precipitated by a variety of alternative methods, including ultrafiltration, ethanol precipitation, conventional or PFGE agarose electrophoresis, etc. It is generally contemplated that the isolated genomic DNA has a purity of about 1.8-1.9, and preferably a purity of about 1.9-2.0 as measured by the OD_{260/280}.

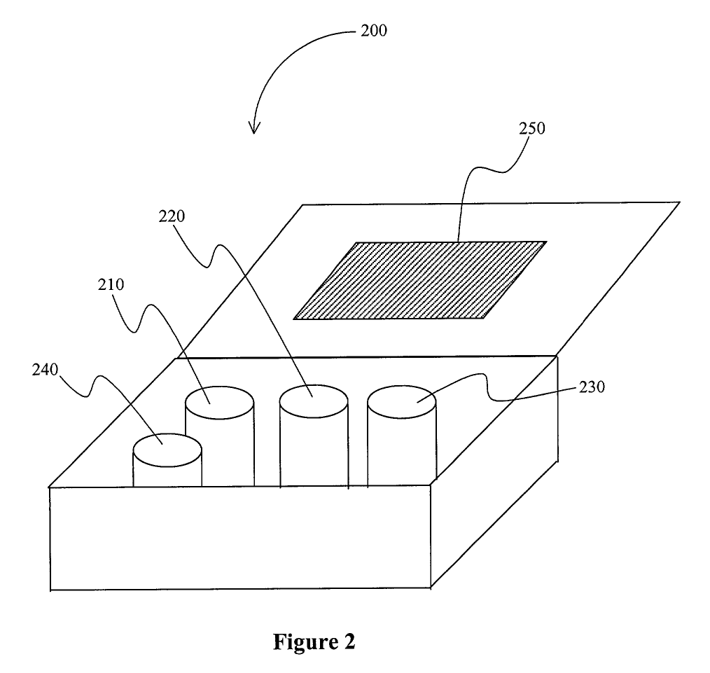

In FIG. 2, a kit 200 for purifying genomic deoxyribonucleic acid from whole blood comprises a first aqueous solution 210 with a first inorganic salt and a second inorganic salt, wherein the first aqueous solution lyses a red blood cells. The kit further comprises a second aqueous solution 220 with a detergent, wherein the second aqueous solution lyses a white blood cell, and a third aqueous solution 230 comprising an organic salt, wherein the third aqueous solution precipitates a protein. Genomic DNA isolated from whole blood following a procedure employing the first, second and third aqueous solution is substantially free of a polymerase inhibitor.

With respect to the blood, the first, second, and third aqueous solutions, and the genomic DNA, the same considerations as described above apply, wherein the second aqueous solution in the kit corresponds to the third aqueous solution as described above, and wherein the third aqueous solution in the kit corresponds to the fourth aqueous solution as described above. Contemplated kits may further comprise isopropanol 240 and/or a printed protocol 250 comprising instructions.

Thus, specific embodiments and applications for purification of genomic DNA from biological fluids have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of isolating genomic deoxyribonucleic acid from whole blood, comprising:
providing a biological fluid comprising a red blood cell and a white blood cell, wherein the white blood cell has a genomic deoxyribonucleic acid and a protein;
lysing the red blood cell by adding a first aqueous solution to the biological fluid, thereby forming a lysis mixture;
removing the white blood cell from the lysis mixture, and washing the white blood cell with a second aqueous solution;
lysing the white blood cell by adding a third aqueous solution to the white blood cell, thereby forming an extraction mixture;
precipitating the protein from the extraction mixture by adding a fourth aqueous solution; and
removing the precipitated protein from the extraction mixture, thereby forming a nucleic acid solution comprising the genomic deoxyribonucleic acid, wherein the nucleic acid solution is substantially free of a polymerase inhibitor.

2. The method of claim 1 wherein the first and second aqueous solutions are the same.

3. The method of claim 1 wherein the first, second, third, and fourth aqueous solution are free of a chaotropic salt and an organic solvent.

4. The method of claim 3 wherein the biological fluid comprises whole blood.

5. The method of claim 3 wherein the first aqueous solution comprises a first inorganic salt and a second inorganic salt.

6. The method of claim 5 wherein the first aqueous solution substantially consists of about 150 mM ammonium chloride, about 10 mM sodium bicarbonate, and about 0.1 mM sodium ethylenediaminetetraacetic acid.

7. The method of claim 3 wherein the step of removing the white blood cell and the step of removing the precipitated protein comprises centrifugation.

8. The method of claim 3 wherein the third aqueous solution comprises a detergent.

9. The method of claim 8 wherein the third aqueous solution substantially consists of about 0.1 wt % sodium lauryl sulfate.

10. The method of claim 3 wherein the fourth aqueous solution comprises an organic salt.

11. The method of claim 10 wherein the fourth aqueous solution substantially consists of 5M ammonium acetate.

12. The method of claim 3 wherein the first and second aqueous solution substantially consist of about 150 mM ammonium chloride, about 10 mM sodium bicarbonate, and about 0.1 mM sodium ethylenediaminetetraacetic acid, the third aqueous solution substantially consists of about 0.1 wt % sodium lauryl sulfate, and the fourth aqueous solution substantially consists of 5M ammonium acetate.

13. The method of claim 3 further comprising precipitating the genomic deoxyribonucleic acid by adding isopropanol to the nucleic acid solution.

14. The method of claim 3 wherein at least one of the steps of providing a biological fluid, lysing the red blood cell, removing the white blood cell, lysing the white blood cell, precipitating the protein, and removing the precipitated protein is automated.

15. A kit for purifying genomic deoxyribonucleic acid from whole blood, comprising:
a first aqueous solution comprising a first inorganic salt and a second inorganic salt, wherein the first aqueous solution lyses a red blood cell;
a second aqueous solution comprising a detergent, wherein the second aqueous solution lyses a white blood cell;
a third aqueous solution comprising an organic salt, wherein the third aqueous solution precipitates a protein; and
wherein a genomic nucleic acid isolated from whole blood following a procedure that employs the first, second and third aqueous solution is substantially free of a polymerase inhibitor.

16. The kit of claim 14 wherein the first aqueous solution substantially consists of about 150 mM ammonium chloride, about 10 mM sodium bicarbonate, and about 0.1 mM sodium ethylenediaminetetraacetic acid.

17. The kit of claim 14 wherein the second aqueous solution substantially consists of about 0.1 wt % sodium lauryl sulfate.

18. The kit of claim 14 wherein the third aqueous solution substantially consists of 5M ammonium acetate.

19. The kit of claim 14 wherein the first aqueous solution substantially consists of about 150 mM ammonium chloride, about 10 mM sodium bicarbonate, and about 0.1 mM sodium ethylenediaminetetraacetic acid, the second aqueous solution substantially consists of about 0.1 wt % sodium lauryl sulfate, and the third aqueous solution substantially consists of 5M ammonium acetate.

20. The kit of claim 14 further comprising isopropanol.

21. The kit of claim 14 further comprising a printed protocol comprising instructions according to claim 1.
